(19) United States
(12) Patent Application Publication
(10) Pub. No.: US 2026/0208137 A1
(43) Pub. Date: Jul. 23, 2026

(54) RADIATIVE HEAT EXCHANGERS FOR TUBE SEGMENTS SUCH AS HEATING TUBES IN ELECTRIC IMPEDANCE FURNACES

(71) Applicant: SABIC GLOBAL TECHNOLOGIES B.V., BERGEN OP ZOOM (NL)

(72) Inventors: Michael Edward HUCKMAN, Bangalore (IN); Robert R. BROEKHUIS, Sugar Land, TX (US)

(73) Assignee: SABIC GLOBAL TECHNOLOGIES B.V., BERGEN OP ZOOM (NL)

(21) Appl. No.: 19/142,950

(22) PCT Filed: Dec. 5, 2023
(86) PCT No.: PCT/EP2023/084297

§ 371 Date: Jun. 24, 2025

(30) Foreign Application Priority Data
Dec. 29, 2022 (EP) 22217071.4

(51) Int. Cl. B01J 8/06; C01B 3/384; C07C 4/04; C10G 9/24; F22B 1/18

(52) U.S. Cl. CPC B01J 8/062; B01J 8/065; B01J 8/067; C01B 3/384; C07C 4/04; C10G 9/24; F22B 1/18; B01J 2208/00407; B01J 2208/00415; B01J 2208/065; C01B 2203/0233; C01B 2203/085

(57) ABSTRACT

This disclosure includes electric impedance furnaces for chemical production processes and methods of heating a feedstock with the present electric furnaces. The present electric furnaces include a primary heating section; a radiative heat exchanger section comprising an exchanger housing with first and second exchanger sidewalls; and a plurality of electrically conductive reactor tubes extending through the heater housing and the exchanger housing, each reactor tube defining a flowpath and having a flanged connector at respective ends of the tube; and an electric circuit configured to electrically connect in series a longitudinal portion of each of the reactor tubes such that application of a voltage difference between a first end of the longitudinal portion of a first one of the reactor tubes and a second end of the longitudinal portion of a last one of the reactor tubes will cause electrical current to flow sequentially through the longitudinal portions of all of the plurality of reactor tubes. In some such furnaces, respective ends of the reactor tubes are electrically isolated from electrically conductive feedstock and collection manifolds, for example by flanged pipe isolation joints. In some such furnaces, one or both of the exchanger sidewalls each define channel configured to receive a fluid to absorb thermal energy from the respective exchanger sidewall. The present methods comprise: heating a feedstock using one of the present furnaces by applying a voltage difference between a first end of the longitudinal portion of a first one of the reactor tubes and a last end of the longitudinal portion of a second one of the reactor tubes to cause electrical current to flow sequentially through the longitudinal portions of all of the plurality of reactor tubes; where the voltage difference is greater than 100 volts (V); and where the current does not flow into either of the collection manifold or the feedstock manifold.

### FIELD OF DISCLOSURE

The present disclosure is generally related to industrial furnaces and, more particularly but not by way of limitation, to a radiative heat exchanger for tube segments, such as heating tubes in impedance furnaces.

### BACKGROUND

Chemical synthesis plants are utilized to provide a variety of chemicals. Often, a dedicated fuel is burned or combusted to provide heat of reaction for chemical synthesis, energy to heat one or more process streams, energy to vaporize liquids (e.g., boil water used as a diluent), energy to do work (e.g., drive a compressor or pump), or energy for other process operations throughout the chemical synthesis plant. Such burning or combustion of fuels results in the production of flue gases that contain CO₂, which can be harmful to the environment, and also results in a loss of energy efficiency of the process. Likewise, steam is often conventionally utilized as a plant-wide heat and/or energy transfer fluid within chemical synthesis plants. The steam utilized for the heat and/or energy transfer is often produced via the combustion of a fuel, resulting in the production of additional flue gas and further energy efficiency losses during the chemical synthesis.

However, the electrification of certain components in chemical synthesis plants presents additional issues and challenges. For example, in steam cracking processes, electrically heated pyrolysis furnaces may present issues or may be subject to considerations that are different than and/or not necessarily present in combustion-driven pyrolysis furnaces. As one example, impedance furnaces typically utilize electric current flowing through the walls of tubes to heat fluids flowing through the tubes, such that the tube becomes both the conduit for the process and the conducting element for electric heating.

However, such impedance furnaces can be economically challenging because the tubes, which are energized electrically, are connected to upstream and downstream piping and/or manifold systems that are not energized. The conventional approach to energizing the tubes in such an electric furnace is to configure the circuit such that the high voltage or potential is at the middle of each tube, and the low voltage or potential (e.g., very close to ground) is at each ends of the tube (near the upstream/downstream connections). In this configuration, energization of the upstream/downstream piping is avoided by causing current to flow into and out of the furnace tubing within the furnace itself. But this arrangement makes each furnace tube its own circuit and, using conventional tube materials of construction (usually various hi alloy metals) the voltage drop across the circuit is very low-typically from 10 Volts (V) to 50 V.

### SUMMARY

With a small voltage drop of 10 V to 50 V, delivering the heating duty of large industrial furnaces—for example, from 10 megawatts (MW) to 250 MW (e.g., 100 MW)—requires enormous current. For example, a 20 MW furnace at 20V would require 1.0 million amps, for which the electrical equipment—such as step down transformers, switch gear, thyristors, hook up panels, copper conductor, and associated components—can be prohibitively expensive. Moreover, the complexity of the electrical equipment required for such massive current introduces heat losses that can make a low-voltage impedance furnace relatively inefficient. As such, higher voltages would be helpful to make impedance furnaces commercially viable.

One way to achieve higher voltage is to electrically insulate the furnace tubes from their upstream/downstream piping connections. With the heater tubes electrically isolated, an electrical circuit can be configured with the furnace tubes connected in series, such that the voltage drop can be much larger, for example, 480 V, 4160 V, or 13,200 V (13.2 kV), which are standard voltages already used (and therefore available) at industrial sites. At these higher voltages, the required current is proportionally less, as are the costs and complexity of the required electrical equipment. For example, a 20 MW furnace at 4160 V requires only 4800 amps (versus 1.0 million amps at 20 V), such that the complexity and cost of required electrical equipment can be significantly reduced relative to that required for much-larger currents and lower voltages. Heat losses are also reduced, such that the high voltage furnace can be significantly more efficient.

Electrical insulation can be achieved with pipe isolation joints (PIJs) that each mechanically couple an end of an electrically conductive furnace tube to an electrically conductive upstream or downstream pipe or manifold, with a sealed connection between their respective flowpaths of the tube and pipe/manifold, and without allowing electrical connection or current flow between the tubes and the pipe/manifold. For example, dielectric or other electrically insulating material can be disposed between the tube and the pipe/manifold to prevent physical contact between their respective materials. Different dielectric or other electrically insulating materials can be selected for different applications. For example, with increasing temperature, at least some dielectrics will undergo changes in the dielectric properties that may being to allow current to pass, for example at higher voltages. Stated another way, with increasing temperature, the ability of a dielectric to withstand a voltage difference without current flow diminishes and eventually breaks down. In addition to appropriate selection of dielectric materials for the PIJ, other characteristics may also be selected to ensure desired electrically insulating properties; for example, increasing the thickness of the dielectric material can increase the breakdown voltage at which the dielectric will begin to allow current to flow (and can therefore increase the temperature at which the breakdown voltage falls below a desired threshold).

The present disclosure includes radiative heat exchangers for industrial-scale impedance furnaces (e.g., for steam cracking, steam methane reforming, and/or various other applications) with pipe isolation joints that electrically isolate segments of furnace tubes and thereby enable the use of higher voltages and improved efficiency.

However, one challenge with pipe isolation joints for electrical isolation of tubing segments is that certain dielectric materials will typically exhibit a breakdown voltage above which the dielectric material begins to conduct electrical current. The breakdown voltage for a particular material may also have an inverse relationship with temperature, meaning that, at elevated temperatures, the breakdown voltage may decrease. Additionally, certain dielectric materials such as electrically insulating polymers exhibit physical changes (e.g., melting) at elevated temperatures, which melting can both frustrate the insulating properties and can permit undesirable leakage of fluids from the joint.

Heating and elevated temperatures are necessary for the tubes of electric impedance furnaces. However, in the present configurations, portions of each tube (downstream of the actively heated portion of the tube) can be cooled after the contents of the tube have been sufficiently heated to undergo desired chemical reactions. Even so, the electrical current flowing longitudinally along each tube in an impedance furnace means that electrical communication must be limited between respective tubes to prevent shorting the heating circuit. As a result, exterior portions of the heating tubes cannot be cooled with water or other electrically conductive liquid in a common chamber because doing so would allow the liquid to conduct electricity between the tubes. However, in the present configurations, downstream portions of such tubes are extended through one or more of the present radiative heat exchangers to reduce the temperature of such tubes between a primary heating section of such a furnace and respective pipe isolation joints (PIJs), such that maximum temperature to which the PIJs are exposed is limited to a range in which the material(s) of the PIJ maintain their physical stability and/or electrically insulating properties.

In some configurations of the present electric furnaces for chemical-production processes, the furnace comprises: a primary heating section; a radiative heat exchanger section; a plurality of electrically conductive reactor tubes; and an electric circuit. In such configurations, the primary heating section comprises a heater housing having a first end, a second end, and first and second furnace sidewalls extending from the first end to the second end; the radiative heat exchanger section comprises an exchanger housing having an exchanger first end, an exchanger second end, and first and second exchanger sidewalls extending from the exchanger first end to the exchanger second end; the reactor tubes extend through the heater housing and the exchanger housing, with each of the reactor tubes defining a flowpath from an inlet end of the reactor tube to an outlet end of the reactor tube, and each of the inlet ends and outlet ends having a flanged connector; and the electric circuit is configured to electrically connect in series a longitudinal portion of each of the reactor tubes such that application of a voltage difference between a first end of the longitudinal portion of a first one of the reactor tubes and a second end of the longitudinal portion of a last one of the reactor tubes will cause electrical current to flow sequentially through the longitudinal portions of all of the plurality of reactor tubes, where at least a majority of the longitudinal portions are disposed in the heater housing. In such configurations, the radiative heat exchanger section is configured to, for each of the reactor tubes, reduce the temperature of the outlet end of the tube relative to the nearest first or second end of the longitudinal portion of the tube.

In some configurations of the present furnaces, the inlet ends of the reactor tubes are disposed on the first end of the heater housing, and the outlet ends of the reactor tubes are disposed on the second end of the exchanger housing.

In some configurations of the present furnaces, the inlet ends and the outlet ends of the reactor tubes are disposed on the second end of the exchanger housing, and each reactor tube includes a U-shaped portion at the first end of the heater housing.

In some configurations of the present furnaces, the plurality of reactor tubes includes a first subset of the reactor tubes and a second subset of the reactor tubes, the first subset of the reactor tubes are disposed with their inlet ends on the first end of the heater housing and their outlet ends on the second end of the exchanger housing, and the second subset of the reactor tubes are disposed with their outlet ends on the first end of the heater housing and their inlet ends on the second end of the exchanger housing.

In some configurations of the present furnaces, the electrical circuit connects the second end of the longitudinal portion of a first one of the reactor tubes to the first end of the longitudinal portion of a second one of the reactor tubes, and connects the second end of the longitudinal portion of the second one of the reactor tubes to the first end of the longitudinal portion of a third one of the reactor tubes.

In some configurations of the present furnaces, (a) the first exchanger sidewall defines a channel configured to receive a fluid to absorb thermal energy from the first exchanger sidewall; (b) the second exchanger sidewall defines a channel configured to receive a fluid to absorb thermal energy from the second exchanger sidewall; or (c) both (a) and (b). In some such configurations, an outlet of each channel(s) is coupled in fluid communication to a steam drum.

Some configurations of the present furnaces, further comprise a catalyst disposed in the reactor tubes, where the catalyst is a steam-reforming catalyst configured to cause steam and methane gas to react to form hydrogen and carbon monoxide.

In some configurations of the present furnaces, (a) the outlet ends of the reactor tubes are coupled to an electrically conductive collection manifold, and where the reactor tubes are electrically isolated from the collection manifold; (b) the inlet ends of the reactor tubes are coupled to an electrically conductive feedstock manifold, and where the reactor tubes are electrically isolated from the feedstock manifold; or (c) both (a) and (b). In some such configurations, the electrical isolation of each reactor tube is provided by one or more flanged pipe isolation joints (PIJs).

Some implementations of the present methods comprise: heating a feedstock using one of the configurations of the present furnaces by applying a voltage difference between a first end of the longitudinal portion of a first one of the reactor tubes and a last end of the longitudinal portion of a second one of the reactor tubes to cause electrical current to flow sequentially through the longitudinal portions of all of the plurality of reactor tubes; where the voltage difference is greater than 100 volts (V); and where the current does not flow into either of the collection manifold or the feedstock manifold.

In some implementations of the present methods, the furnace is part of a steam methane reformer (SMR) system, and the feedstock comprises steam and methane.

In some implementations of the present methods, the furnace is part of a steam cracking system, and the feedstock comprises steam and at least one component selected from the list of components consisting of: naphtha, liquefied petroleum gas (LPG), and ethane.

Some implementations of the present methods further comprise: passing H₂O through the channel(s) of the exchanger sidewall(s) to absorb thermal energy from the exchanger sidewall(s). Some such implementations further comprise: delivering the H₂O from the channel(s) of the exchanger sidewall(s) to a steam drum to separate liquid and steam from the H₂O.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any embodiment of the present apparatuses, kits, and methods, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, an apparatus, device or system that is configured in a certain way is configured in at least that way, but it can also be configured in other ways than those specifically described.

Any embodiment of any of the present apparatuses and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

Some details associated with the aspects of the present disclosure are described above, and others are described below. Other implementations, advantages, and features of the present disclosure will become apparent after review of the entire application, including the Brief Description of the Drawings, Detailed Description, and the Claims.

### BRIEF DESCRIPTION OF THE DRAWINGS

The following drawings illustrate by way of example and not limitation. For the sake of brevity and clarity, every feature of a given structure is not always labeled in every figure in which that structure appears. Identical labels or reference numbers do not necessarily indicate an identical structure. Rather, the same reference number may be used to indicate a similar feature or a feature with similar functionality, as may non-identical reference numbers. Dimensioned figures are drawn to scale (unless otherwise noted), meaning the sizes of the depicted elements are accurate relative to each other for at least the embodiment depicted in the figures.

FIG. 1 depicts a block flow diagram of a generalized methanol synthesis plant or process.

FIG. 2 depicts a block diagram of an electric impedance furnace that may be configured for use in the syngas synthesis section of the plant or process of FIG. 1.

FIG. 3 depicts a conceptual diagram of a first example of an impedance furnace for use in the pyrolysis reaction section of FIG. 2.

FIG. 4 depicts a cross-sectional side view of a first example of the present pipe isolation joints for use in impedance furnaces such as the example of FIG. 3.

FIG. 5 depicts a plan view of an insulating disc for use with the pipe isolation joint of FIG. 4.

FIG. 6A depicts a conceptual diagram of a second example of an impedance furnace with two radiative heat exchangers for use in the pyrolysis reaction section of FIG. 2.

FIG. 6B depicts a cross-sectional view of one of the radiative heat exchangers of FIG. 6A, taken along the line 6B-6B of FIG. 6A.

FIG. 7A depicts a conceptual diagram of a third example of an impedance furnace with a radiative heat exchanger for use in the pyrolysis reaction section of FIG. 2.

FIG. 7B depicts a cross-sectional view of the radiative heat exchanger of FIG. 7A, taken along the line 7B-7B of FIG. 7A.

### DETAILED DESCRIPTION

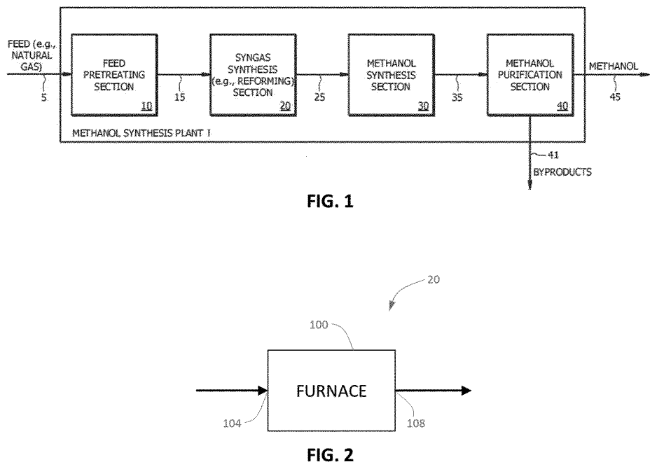

Referring now to the drawings, and more particularly to FIG. 1, shown there is a block flow diagram of an example of a generalized methanol synthesis plant, which includes one or more of the following process sections for converting a feed stream 5 into a methanol product stream 45 (and optionally one or more byproduct streams 41): a feed pretreating section 10, a syngas synthesis section 20, a methanol synthesis section 30, a methanol purification section 40, or a combination thereof. Such sections will be described briefly in the next few paragraphs, and in more detail hereinbelow.

As indicated in the methanol synthesis block flow diagram, a feed pretreating section 10 of a methanol synthesis plant is operable to prepare (e.g., remove undesirable components (e.g., sulfur) from, adjust temperature and/or pressure of) a feed 5 for reforming, providing a pretreated feed 15. In some applications, a methanol synthesis plant of this disclosure does not comprise a feed pretreating section. A syngas synthesis section 20 is operable to produce synthesis gas from feed 5 or pretreated feed 15 to produce a syngas synthesis product 25 comprising carbon monoxide (CO) and hydrogen (H₂).

In some implementations, syngas generation section 20 is a syngas synthesis section operable to carry out steam reforming of the feed (e.g., of a feed 5 or pretreated feed 15 comprising natural gas) to produce a reformer product comprising carbon monoxide (CO) and hydrogen (H₂). The syngas synthesis (or ‘reformer’) product 25 can further comprise carbon dioxide (CO₂), water, methane (CH₄), and/or impurities. For example, some embodiments of the present electric furnaces can comprise a steam reforming catalyst to carry out the syngas reaction to produce carbon monoxide and hydrogen.

A methanol synthesis section 30 is operable to produce methanol from the syngas synthesis product 25 and thus provide a crude methanol stream 35. A methanol purification section 40 is operable to separate a purified methanol product 45 and byproducts 41 from the crude methanol stream 35.

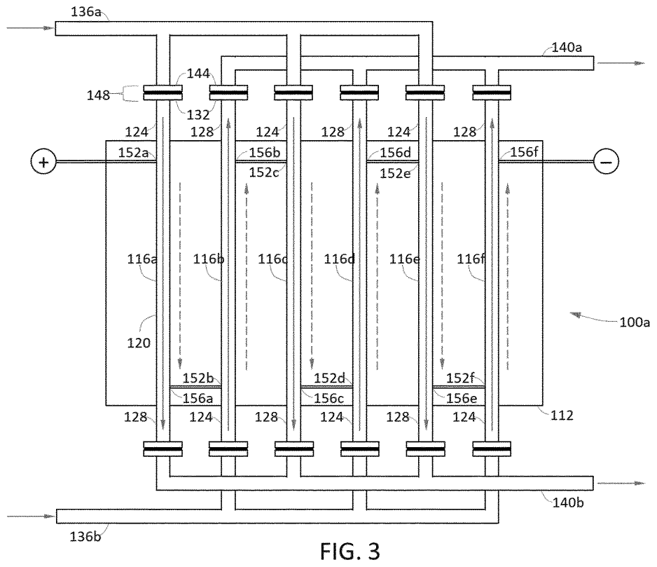

Referring now to FIG. 2, shown there is a block diagram of an electric impedance heater 100 that may be configured for use in the syngas synthesis (steam reforming) section 20 of the plant or process of FIG. 1. As shown in FIG. 2, the furnace generally includes a fluid inlet 104 and a fluid outlet 108.

FIGS. 1 and 2 depict one example of a methanol synthesis system for illustration purposes, but the present electric impedance furnaces can be used in any of various chemical synthesis systems and processes, particularly in place of furnaces that have historically been powered by direct combustion or fossil fuels.

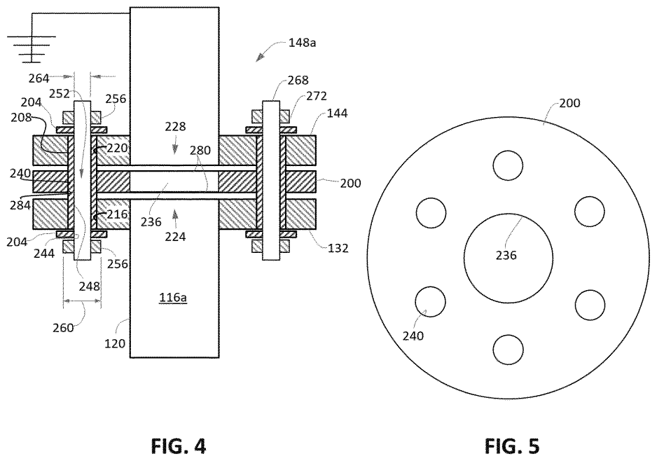

Referring now to FIG. 3, shown there is a conceptual diagram of a first example 100a of an impedance furnace for use in the pyrolysis reaction (20) section of FIG. 2. In this configuration, furnace 100a comprises a housing 112 and a plurality of furnace tubes 116 extending across the housing. Each tube 116 includes a sidewall 120 defining a flow channel extending from an inlet end 124 to an outlet end 128, each of which including a flange 132.

In the depicted configuration, furnace 100a is coupled to first and second inlet manifolds 136a, 136b, and first and second outlet manifolds 140a, 140b. Specifically, inlet ends 124 of the tubes (116) are each coupled to a respective one of inlet manifolds 136a, 136b; and outlet ends 128 of the tubes (116) are each coupled to a respective one of outlet manifolds 140a, 140b. Each inlet manifold 136a, 136b includes a plurality of connections with flanges 144 coupled to respective flanges 132 via pipe isolation joints 148 at inlet ends 124 of tubes 116; and each outlet manifold 140a, 140b includes a plurality of connections with flanges 144 coupled to respective flanges 132 via pipe isolation joints 148 at outlet ends 128 of tubes 116. As described in more detail below, pipe isolation joints 148 electrically insulate the furnace tubes 116 from the manifolds (136a, 136b, 140a, 140b) and therefore prevent electric current from flowing between the tubes and the manifolds.

Because furnace tubes 116 are electrically isolated from the manifolds (136a, 136b, 140a, 140b), an electric potential can be applied across multiple furnace tubes in series. For example, in the depicted configuration—which includes a relatively small number of tubes 116 for illustration purposes—the tubes are electrically connected in series to a voltage difference applied across all of the tubes in series so that a voltage difference applied across the tubes causes current to flow sequentially through each of the tubes. Specifically, a point 156a near outlet end 128 of a first tube 116a is electrically connected to a point 152b nearer inlet end 124 of a second tube 116b, a point 156b nearer outlet end 128 of second tube 116b is electrically connected to a point 152c nearer inlet end 124 of a third tube 116c, a point 156c nearer outlet end 128 of third tube 116c is electrically connected to a point 152d nearer inlet end of a fourth tube 116d, a point 156d nearer outlet end 128 of fourth tube 116d is electrically connected to a point 152e nearer inlet end 124 of fifth tube 116e, and a point 156e nearer outlet end 128 of fifth tube 116e is electrically connected to a point 152f nearer inlet end 124 of a sixth tube 116f. As such, when a voltage difference is applied across the furnace tubes (116) with the high potential (represented by the + circle) at point 152a nearer inlet end 124 of that first tube 116a, and the low potential (represented by − circle) at point 156f nearer outlet end 128 of sixth tube 116f, then current flows sequentially through point 152a, point 156a, point 152b, point 156b, point 152c, point 156c point 152d, point 156d, point 152e, point 156e, point 152f, and point 156f, as indicated by the dashed arrows next to the tubes (116). In this configuration, points 152 and 156 are all disposed within the furnace housing (112) so that substantially all current flowing along the tubes (116) remains within furnace housing.

While a small number of furnace tubes 116 is shown for illustration purposes, industrial furnaces will typically include a greater number of tubes through which fluids can flow and be heated, such that the voltage drop along each tube will typically be on the order of 50 V. For example, a furnace with a voltage drop across all tubes 116 of 4160 V and eighty (80) furnace tubes 116 will exhibit an average voltage drop of roughly 50 V per tube. Thus, the electrical isolation of tubes 116 from the upstream and downstream piping or manifolds (e.g., 136a, 136b, 140a, 140b) and resulting ability to connect tubes in sequence allows a much greater voltage drop overall, dramatically reducing the current required to generate heating duties sufficient for industrial applications. For example, a 20 MW impedance heater with a voltage of 4160 V requires only 4800 amps (versus 1 million amps for a similar heater with a voltage of only 20 V).

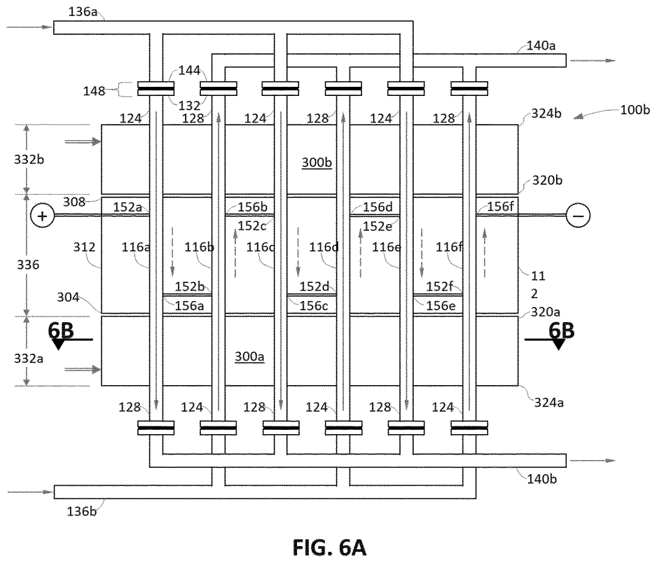

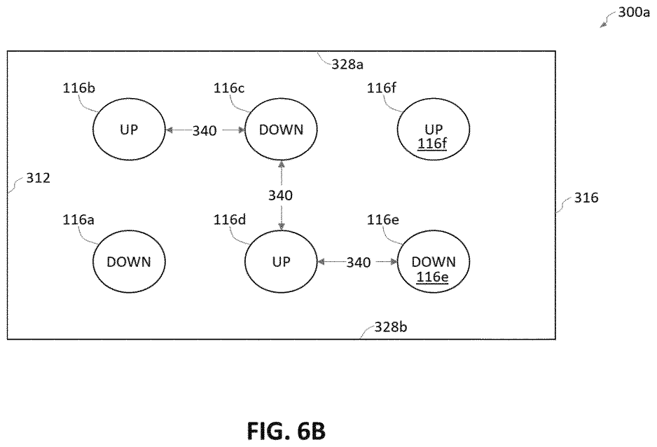

Referring now to FIGS. 4 and 5, FIG. 4 depicts a cross-sectional side view of a first example 148a of the present pipe isolation joints for use in impedance furnaces such furnace 100a, and FIG. 5 depicts a plan view of an insulating disc for use with pipe isolation joint 148a. In the depicted configuration, pipe isolation joint 148a is an assembly that comprises an electrically conductive first flange 132, an electrically conductive second flange 144, an insulating disc 200, a plurality of fastener insulators 204, one or more insulating sleeves 208, and one or more fastener assemblies 212.

Flange 132 defines a first opening 216, and flange 144 defines a second opening 220 that is configured to be aligned with first opening 216 as shown. In the depicted configuration, flange 132 defines a plurality of first openings 216 surrounding a primary first passage 224 (similar to the insulating disc of FIG. 5), and second flange 144 defines a plurality of second openings 220 surrounding a primary second passage 228 such that passage 228 is configured to align with passage 224, and each of second openings 220 is configured to align with a corresponding one of first openings 216.

Insulating disc 200 comprises an electrically non-conductive material and defines one or more disc openings 240. For example, in the depicted example, insulating disc 200 defines a primary disc passage 236 and a plurality of disc openings 240 surrounding and spaced from one another (e.g., at equiangular intervals) primary disc passage 236, such that primary disc passage 236 is configured to align with primary first and second passages 224, 228, and disc openings 240 are configured to align with respective ones of first and second openings 216, 220. Primary disc passage 236, as well as primary first passage 224 and primary second passage 228 (and their respective tubes or pipes), can have an inner diameter of from 1 inch to 10 inches (e.g., between any two of 1 inch, 2 inches, 3 inches, 4 inches, 5 inches, 6 inches, 7 inches, 8 inches, 9 inches, and/or 10 inches). For example, such inner diameter is between 1 inches and 3 inches in some configurations, and between 5 inches and 7 inches in other configurations.

Each fastener insulator 204 comprises an electrically non-conductive material and defines one or more fastener holes 244. For example, in the depicted configuration, each fastener insulator 204 has a conventional washer shape defining a single fastener hole 244; however, in other configurations, each fastener insulator 204 can be shaped as a disc defining a plurality of fastener holes similar to the insulating disc of FIG. 5.

Each insulating sleeve 208 comprises an electrically non-conductive material and defines an inner fastener channel 248. Additionally, and as shown in FIG. 4, each insulating sleeve 208 has an outer profile that is configured to extend through a first opening 216 of flange 132 and a corresponding second opening 220 of flange 144, and into (e.g., and through) disc opening 240 of insulating disc 200.

Each fastener assembly 212 includes a longitudinal medial portion 252, and first and second retention portions 256 each having a transverse dimension 260 that is larger than a corresponding transverse dimension 264 of the medial portion. In at least some configurations, fastener assemblies 212 each comprise electrically conductive material such as steel or another metallic alloy. In the depicted configuration, each fastener assembly comprises a threaded stud 268 with nuts 272 threaded onto opposing ends of the stud, as shown. In other configurations, each fastener assembly can have any configuration that permits the described functionality. For example, in other configurations, each fastener assembly can include a bolt with an enlarged head on one end, and a nut 272 threaded onto the opposing end.

As shown in FIG. 4, flange 132 is configured to be coupled to flange 144 with insulating disc 200 between the flanges. In use, each insulating sleeve 208 extends through a first opening 216 of flange 132, through a corresponding second opening 120 of flange 144, and into (e.g., through) a corresponding disc opening 240. A respective fastener assembly 212 also extends through the insulating sleeve (208) with one of the fastener insulators (204) disposed between the first retention portion 256 and flange 132, and another one of the fastener insulators (204) disposed between the second retention 256 portion and flange 148.

As shown, insulating disc 200, insulating sleeve(s) 208, and fastener insulators 204 are configured such that, once assembled, they prevent the fastener assembly from contacting either flange and prevent flanges 132, 144 from contacting each other, and thereby electrically isolate flange 132 from flange 144.

The electrically nonconductive material of each of insulating disc 200, insulating sleeve(s) 208, and fastener insulator(s) 204 is selected to remain solid and electrically nonconductive at the desired operating temperatures and voltages, for example, at voltages up to 200 V and temperatures up to 300° C. In some configurations, the electrically nonconductive material of insulating disc 200, insulating sleeve(s) 208, and/or fastener insulator(s) is selected to remain solid and electrically nonconductive at voltages above 100V (e.g., above any one of or between any two of: 100 V, 200 V, 300 V, 400 V, 500 V, 600 V, 700 V, 800 V, 900 V, and/or 1000 V) and temperatures in excess of 300° C. (e.g., above any one of or between any two of: 300° C., 400° C., 500° C., 600° C., or more). In some implementations, such as those in which the isolation disc is particularly rigid and resists deforming sufficiently under pressure to seal the interface, an additional sealant (that need not be electrically insulative) is added between isolation disc 200 and the respective flanges (132, 144).

Various polymers (e.g., polyetherimide (PEI)), copolymers (e.g., of PEI), and ceramics (e.g., Alumina, Zirconia, Silicon Nitride, Tricalcium Phosphate, and silica-based materials) can be configured to exhibit these characteristics, and are sufficiently shapeable (e.g., via molding, machining, and/or other methods) to be provided with at least some of the shapes described in this disclosure for the insulating disc, insulating sleeves, and fastener insulators. Some such electrically nonconductive materials, such as polymers, may exhibit physical changes, such as melting, with increasing temperature on the order of 300° C. to 400° C. Some PEIs, for example, exhibit melting temperatures as high as 340° C. to 360° C. As such, as long as it is chemically stable in the present of fluids to which it will be exposed, PEI can be a suitable electrically nonconductive material for desired operating temperatures on the order of 250° C. or 275° C. Other materials (e.g., polymers) remain electrically insulative and dimensionally stable at higher temperatures, for example, on the order of 500° C., 550° C., or even 600° C. or more.

For voltages on the order of 100 V and greater (e.g., 1000 V) across the pipe isolation joint, the minimum thickness of the nonconductive material of the isolation disc (200), the fastener insulators (204), and the insulating sleeve(s) 208—for example, the vertical thickness of each of the isolation disc (200) and the fastener insulators (204), and the horizontal thickness of the sidewall of the insulating sleeve—can be 0.15 inches or larger (e.g., larger than any one of or between any two of: 0.15 inches, 0.20 inches, 0.25 inches, 0.30 inches, 0.35 inches, 0.40 inches, 0.45 inches, 0.50 inches, or larger). In some configurations, the thickness of the isolation disc is greater than the thickness of each the fastener insulators (204) and/or greater than the thickness of the sidewall of the insulating sleeve (208). For example, in some configurations, the fastener insulators (204) and insulating sleeve (208) each have a first minimum thickness (e.g., larger than any one of or between any two of: 0.15 inches, 0.20 inches, 0.25 inches, 0.30 inches, 0.35 inches, 0.40 inches, 0.45 inches, 0.50 inches, or larger); and the isolation disc (200) has a second minimum thickness (e.g., larger than any one of or between any two of: 0.20 inches, 0.25 inches, 0.30 inches, 0.35 inches, 0.40 inches, 0.45 inches, 0.50 inches, or larger) that is larger than the first minimum thickness. By way of specific example, in one such configuration, the first minimum thickness is 0.25 inches, and the second minimum thickness is 0.5 inches. In addition to directly preventing contact between electrically conductive surfaces, the configuration of the isolation disc (200) and insulating sleeve (208) also ensure spacing between electrically conductive surfaces and the isolation disc (200) and insulating sleeve (208) can be configured to ensure sufficient minimum spacing—an air gap—to avoid electrical arcing between portions of electrically conductive surfaces that are not directly separated or interposed by a portion of either the isolation disc (200) or insulating sleeve (208).

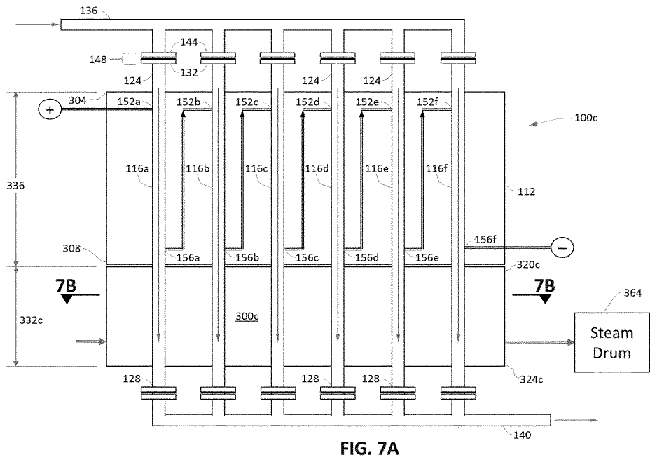

Referring now to FIGS. 6A and 6B, FIG. 6A depicts a conceptual diagram of a second example 100b of an impedance furnace with two radiative heat exchangers 300a, 300b for use in the pyrolysis reaction section (20) of FIG. 2; and FIG. 6B depicts a cross-sectional view of radiative heat exchanger 300a of FIG. 6A, taken along the line 6B-6B. Furnace 100b is similar in many respects to furnace 100a of FIG. 3. For example, as explained above for furnace 100a with reference to FIG. 3, furnace 100b comprises an electric circuit configured to electrically connect in series a longitudinal portion (between 152, 156) of each of the reactor tubes (116) such that application of a voltage difference between a first end 152a of the longitudinal portion of a first one 116a of the reactor tubes and a second end 156f of the longitudinal portion of a last one 116f of the reactor tubes will cause electrical current to flow sequentially through the longitudinal portions of all of the plurality of reactor tubes, where at least a majority (e.g., all, as shown) of the longitudinal portions are disposed in the heater housing (112).

However, furnace 100b also differs from furnace 100a in that furnace housing 112 of furnace 100b includes a first radiative heat exchanger 300a on a first end 304 of furnace housing 112, and a second radiative heat exchanger 300b on an opposing second end 308 of furnace housing 112. Furnace sidewalls 312 extend from first end 304 to second end 308 as shown. In this configuration, each radiative heat exchanger section 300a, 300b is configured to, for each of the reactor tubes, reduce the temperature of the outlet end (128) of the tube relative to the nearest first or second end (e.g., 156b) of the longitudinal portion of the tube. As described in the Summary above, the reduction of temperature (relative to the temperature as the hot side of the tube exits furnace housing 112) of the tube near the flanges 132 at the outlet ends (128) of the tubes can preserve the physical stability and electrically insulative properties of the materials in the respective pipe isolation joints (PIJs) 148.

First radiative heat exchanger 300a comprises an exchanger housing 316 having an exchanger first end 320a, an exchanger second end 324a, and first and second exchanger sidewalls 328a, 328b (FIG. 6B) extending a length 332a from first end 320a to second end 324a. Similarly, second radiative heat exchanger 300b comprises an exchanger housing 316b having an exchanger first end 320b, an exchanger second end 324b, and first and second exchanger sidewalls 328a, 328b (similar to what is shown in FIG. 6B for exchanger 300a) extending a length 332b from first end 320b to second end 324b. In the depicted embodiment, tubes 116 traverse a length 336 within furnace housing (112). As shown in FIG. 6B, tubes 116a-116f are arranged in two rows such that the tubes do not contact each other within the exchangers, and are separated from each other by substantially equal distances 340. In other configurations, the tubes may be arranged in a single row or in more than two rows.

In the depicted embodiment, furnace sidewalls 312 comprise a material with relatively high emissivity (e.g., 0.5 or greater, 0.7 or greater, or 0.8 or greater) such as any of various metal alloys. In some configurations, interior surfaces of at least some of sidewalls 312, and/or exterior surfaces of at least the portions of tubes 116 in exchangers 300a and 300b, are polished, coated, or both to increase emissivity. The relatively high emissivity of sidewalls 312 contributes to the ability of the sidewalls to absorb and emit thermal radiation from and to the tubes 116 within each radiative heat exchanger. For example, as illustrated in FIG. 6B, the depicted configuration includes furnace reactor tubes flowing in alternating directions. Additionally, the exchanger sidewalls (312) will, overall, increase in average temperature due to the net absorption of thermal radiation. As such, to further increase the net cooling of the tubes carrying heated fluids away from the furnace housing (112), in some configurations, the material of furnace sidewalls 312 is also selected to have a relatively high specific heat (e.g., 400 J/kg-K or greater, 450 J/kg-K or greater, or 500 J/kg-K or greater) to increase the thermal energy the sidewalls (312) themselves can temporarily “hold” as thermal energy is dissipated.

In the orientation of FIG. 6B, tubes with fluid flowing down carry fluids that have been heated in, and are being carried away from, the furnace housing (112), whereas tubes with fluid flowing up carry fluids that have not yet been heated and are being carried toward the furnace housing (112). As such, in radiative heat exchanger 300a of FIG. 6B, the “DOWN” tubes are generally hotter emitters of thermal radiation, the “UP” tubes are generally cooler absorbers of thermal radiation, and portions of sidewalls 312 may both absorb and emit thermal radiation depending on temperature and positioning of relative to the tubes. While radiative heat exchange is complex, the net effect of this configuration is to: lower the temperature of the “DOWN” tubes as distance from the furnace housing (112) increases, and thereby lower the temperature of fluid flowing in the “DOWN” tubes as the fluid flows away from the furnace housing (112); and raise the temperature of the “UP” tubes as distance from the furnace housing (112) decreases, and thereby increase the temperature of fluid flowing in the “UP” tubes as the fluid flows toward the furnace housing (112). In operation, radiative heat exchangers 300a, 300b extract thermal energy in the form or thermal radiation from the “hot” fluids exiting the furnace, and use that thermal energy to preheat the “cold” fluids as they flow toward the furnace.

Certain parameters may be varied to impact the degree of cooling the “hot” tubes experience as they extend away from furnace housing to the distal end of the respective heat exchanger. For example, for radiative heat exchanger 300a, length 332a of exchanger 300a may be increased to increase cooling of the “hot” tubes (116a, 116c, 116e) and/or the temperature of the “cold” tubes 116b, 116d, 116f at exchanger second end 328a may be reduced to increase the magnitude of cooling experienced by the “hot” tubes (116a, 116c, 116d). Furnace 100b was modeled for a steam-methane reforming (SMR) process with length 336 of 13 meters (m), and lengths 332a and 332b each equal to 5 meters. With a “cold” gas inlet temperature at the outer ends of the heat exchangers (300a, 300b) of 650° C., and a “hot” gas exiting the furnace at a temperature of 875° C., the “hot” gas decreased in temperature from 875° C. to 848° C. at the outer ends (324a, 324b) of the respective radiative heat exchangers 300a, 300b, and the “cold” gas increased in temperature from 650° C. to 674° C. at the inner ends (320a, 320b) of the respective heat exchangers 300a, 300b. As a result, the temperature at the inner diameter (ID)—i.e., the highest temperature along the thickness of the tube wall—of the “hot” tubes decreased from 866° C. at the inner ends (320a, 320b) of the heat exchangers (the temperature that would be closest to the “hot” tube PIJs 148 in the absence of the present radiative heat exchangers) to 788° C. at the outer ends (324a, 324b) of the heat exchangers.

With lengths 332a and 332b each increased from 5 meters to 10 meters, and all other things being equal, the “hot” gas decreased in temperature from 875° C. to 820° C. at the outer ends (324a, 324b) of the respective radiative heat exchangers 300a, 300b, and the “cold” gas increased in temperature from 650° C. to 699° C. at the inner ends (320a, 320b) of the respective heat exchangers 300a, 300b. As a result, the temperature at the inner diameter (ID)—i.e., the highest temperature along the thickness of the tube wall—of the “hot” tubes decreased from 866° C. at the inner ends (320a, 320b) of the heat exchangers (the temperature that would be closest to the “hot” tube PIJs 148 in the absence of the present radiative heat exchangers) to 770° C. at the outer ends (324a, 324b) of the heat exchangers.

With lengths 332a and 332b each equal to 10 meters, the system was then modeled for a “cold” gas inlet temperature at the outer ends of the heat exchangers (300a, 300b) of 450° C.

With this option, the “hot” gas decreased in temperature from 875° C. to 811° C. at the outer ends (324a, 324b) of the respective radiative heat exchangers 300a, 300b, and the “cold” gas increased in temperature from 450° C. to 552° C. at the inner ends (320a, 320b) of the respective heat exchangers 300a, 300b. As a result, the temperature at the inner diameter (ID)—i.e., the highest temperature along the thickness of the tube wall—of the “hot” tubes decreased from 866° C. at the inner ends (320a, 320b) of the heat exchangers (the temperature that would be closest to the “hot” tube PIJs 148 in the absence of the present radiative heat exchangers) to 695° C. at the outer ends (324a, 324b) of the heat exchangers.

With lengths 332a and 332b each equal to 10 meters, the system was then modeled for a “cold” gas inlet temperature at the outer ends of the heat exchangers (300a, 300b) of 200° C. With this option, the “hot” gas decreased in temperature from 875° C. to 721° C. at the outer ends (324a, 324b) of the respective radiative heat exchangers 300a, 300b, and the “cold” gas increased in temperature from 200° C. to 341° C. at the inner ends (320a, 320b) of the respective heat exchangers 300a, 300b. As a result, the temperature at the inner diameter (ID)—i.e., the highest temperature along the thickness of the tube wall—of the “hot” tubes decreased from 866° C. at the inner ends (320a, 320b) of the heat exchangers (the temperature that would be closest to the “hot” tube PIJs 148 in the absence of the present radiative heat exchangers) to 630° C. at the outer ends (324a, 324b) of the heat exchangers.

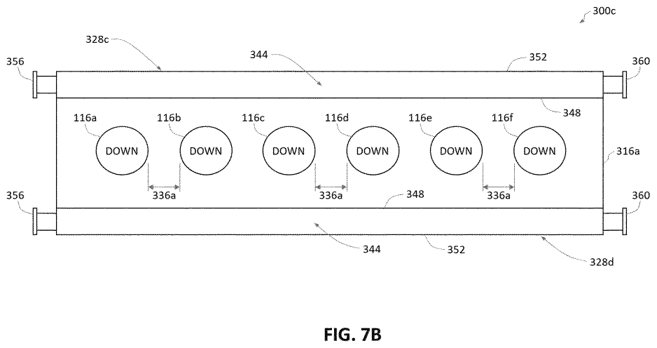

Overall, while increasing the lengths (332a, 332b) of the radiative heat exchanges can lower the tubing temperature at the outer ends (324a, 324b) of the radiative heat exchangers (and therefore for the “hot” tub PIJs 148), reducing the inlet temperature of the “cold” gas has a greater impact on reducing tubing temperature at the outer ends (324a, 324b) of the radiative heat exchangers (and therefore for the “hot” tub PIJs 148). And, while the depicted configuration may be functional for SMR, and even preferable for other processes, it is generally desirable to have a higher “cold” gas inlet temperature for SMR processes, such that the additional configuration of FIGS. 7A and 7B may be preferable for at least some implementations of SMR processes.

Referring now to FIGS. 7A and 7B, FIG. 7A depicts a conceptual diagram of a third example 100c of an impedance furnace and a radiative heat exchanger 300c for use in the pyrolysis reaction section (20) of FIG. 2; and FIG. 7B depicts a cross-sectional view of radiative heat exchanger 300c of FIG. 7A, taken along the line 7B-7B. Furnace 100c is similar in many respects to furnace 100b of FIGS. 6A-6B. For example, as explained above for furnace 100b, furnace 100c comprises an electric circuit configured to electrically connect in series a longitudinal portion (between 152, 156) of each of the reactor tubes (116) such that application of a voltage difference between a first end 152a of the longitudinal portion of a first one 116a of the reactor tubes and a second end 156f of the longitudinal portion of a last one 116f of the reactor tubes will cause electrical current to flow sequentially through the longitudinal portions of all of the plurality of reactor tubes, where at least a majority (e.g., all, as shown) of the longitudinal portions are disposed in the heater housing (112).

However, furnace 100c also differs from furnace 100b in that fluid flows in a common direction through furnace housing 312 (instead of alternating directions as in furnace 100b) such that first end 304 is a “cold” end, and second end 308 is a “hot” end, and furnace 100c includes one radiative heat exchanger 300c on the hot end (308) as opposed to two radiative heaters on opposing ends as in furnace 100b. Accordingly, first ends 124 of all of the tubes (116a, 116b, 116c, 116d, 116e, 116f) are connected to a common inlet manifold 136, and second ends 128 of the tubes are all connected to a common outlet manifold 140, such that fluid flows in the same direction through all of the tubes (downward in the depicted orientation). Additionally, as shown in FIG. 7B, tubes 116 of furnace 100c are arranged in a single row within exchanger housing 316a.

In the depicted configuration, housing 316a has an exchanger first end 320c, an exchanger second end 324c, and first and second exchanger sidewalls 328c, 328d (FIG. 6B) extending a length 332c from first end 320c to second end 324c. In the depicted embodiment, tubes 116 traverse a length 336 within furnace housing (112). As shown in FIG. 6B, tubes 116a-116f are arranged in two rows such that the tubes do not contact each other within the exchangers, and are separated from each other by substantially equal distances 340a. In other configurations, the tubes may be arranged in multiple rows.

Additionally, to facilitate radiative cooling of the tubes in exchanger 300c, sidewalls 328c, 328d each define a channel 344 configured to receive a fluid to absorb thermal energy from the first exchanger sidewall. More particularly, each sidewall 328c, 328 includes an inner wall 348 and an outer wall 352 to define the substantially hollow interior that defines channel 344. In the depicted embodiment, inner sidewalls 348 comprise a material with relatively high emissivity (e.g., 0.5 or greater, 0.7 or greater, or 0.8 or greater) such as any of various metal alloys. In some configurations, interior surfaces of at least some of inner sidewalls 348, and/or exterior surfaces of at least the portions of tubes 116 in exchanger 300c, are polished, coated, or both to increase emissivity. The relatively high emissivity of inner sidewalls 348 contributes to the ability of the sidewalls to absorb and emit thermal radiation from and to the tubes 116 within each radiative heat exchanger. Absent cooling by a fluid within channels 344, the exchanger sidewalls (312) would, overall, increase in average temperature due to the net absorption of thermal radiation. Ideally, the material of each inner sidewall 348 is selected to have both a relatively high emissivity for efficient absorption of thermal radiation from the tubes (116) on the inner surface of inner wall 348, and relatively high conductivity to efficiently transfer thermal energy through its outer surface to the cooling fluid within channel 344.

An inlet 356 is coupled to a first side of housing 316a such that the inlet is in fluid communication with the channel, and an outlet 360 is coupled to a second side of housing 316b such that the outlet is in fluid communication with the channel. A source of cooling fluid can thus be coupled to inlets 356 to pass cooling fluid through the channels and out of outlets 360. When delivered to inlets 356 at a temperature below that of inner walls 348, such a cooling fluid can absorb thermal energy directly from inner walls 348 and thereby lower the temperature of inner wall 348, thereby increasing its absorption of thermal radiation from tubes 116 and increasing the radiative cooling of the tubes (relative to plate walls without additional fluid cooling as in furnace 100b).

In one particular example of a cooling fluid, fluid H₂O, such as boiler feedwater (BFW), can be delivered in a liquid state or in two-phase state (with some liquid and some steam). As the fluid H₂O passes through channels 344, all or some may be converted to steam before reaching outlets 360. When part of the fluid H₂O remains in a liquid state, outlets 360 can be coupled to a steam drum 364 to separate the liquid portion from the gas portion and, for example, the liquid portion can be recirculated to inlets 356 and/or the steam portion can be redirected for recapture of thermal energy elsewhere (e.g., preheating a feed to the reactor tubes 116).

The configuration of FIGS. 7A and 7B was modeled for a steam-methane reforming (SMR) process in which gas in tubes 116 entered the exchanger housing 316a at end 320c at a temperature of 850° C., with boiler feedwater (BFW) delivered to inlets 356 in a liquid state at a flowrate at which part of the BFW vaporized within the channels 344 of the sidewalls 328c, 328d at a typical steam pressure of 600 psi and inner wall 348 temperature of 274° C. Modeling was performed for different heat exchanger lengths 332c, in particular 20 meters, 14 meters, and 10 meters. Results are shown in TABLE 1. “Corner tube” refers to the tubes at the left-most and right-most positions, respectively, in exchanger 300c (see FIG. 7B), which experience higher temperatures due to having a “view” of a smaller portion of the exchanger sidewalls.

TABLE 1

| | | Avg. temp | | | | | | |
|---|---|---|---|---|---|---|---|---|
| | | [C.] of gas | | Peak temp [C.] | | | | |
| | Peak temp | in tubes 116 | | of tubes 116 | | | | |
| Box | [C.] of gas | at end 324a | | at end 324c | | Cooling | Area of | |
| height | in tubes 116 | Corner | Other | Corner | Other | duty | wall 348 | Heat flux |
| [m] | at end 324a | tube | tubes | tube | tubes | [kW] | [m²] | [kW/m²] |
| 20 | 544 | 527 | 511 | 461 | 446 | 492.5 | 74.7 | 6.59 |
| 14 | 613 | 590 | 575 | 502 | 487 | 397.4 | 52.3 | 7.60 |
| 10 | 672 | 643 | 631 | 534 | 518 | 316.1 | 37.3 | 8.47 |

As shown in in TABLE 1, flowing a cooling fluid through the channels (344) of heat exchanger 300c results in significantly larger reductions in temperature of and in the tubes (116) along length 332c of the heat exchanger (relative to that of exchangers 300a, 300b).

While radiative heat exchanger 300c includes two hollow sidewalls 328c, 328d each defining one or more channels 344, other configurations of the present radiative heat exchangers can include only a single hollow sidewall defining one or more channels for cooling fluid. For example, in other configurations of furnace 100a, the reactor tubes may be U-shaped such that the inlet end and outlet end of each tube are disposed on the same end of the furnace. In such a configuration, the “cold” sides of all of the U-shaped tubes can be disposed on a first side of the heat exchanger, and the “hot” sides of all of the U-shaped tubes can be disposed on the opposing second side of the heat exchanger, with a hollow sidewall disposed on the same side as the “hot” sides of the tubes. In such a configuration, passing fluid through the hollow sidewall can increase the magnitude of radiative cooling of the “hot” sides of the tubes, while also allowing for higher inlet temperatures in the “cold” sides of the tubes, which can still receive some degree of radiative preheating from the “hot” sides of the tubes.

Additional details about various components of syngas synthesis plants and processes can be found in International Patent Application Publication No. WO2020/150247, which is incorporated by reference in its entirety.

Additional details about various components of steam cracking plants and processes can be found in International Patent Application Publication No. WO2020/150244, which is incorporated by reference in its entirety.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An electric furnace for a chemical-production process, the furnace comprising:
a primary heating section comprising a heater housing having a first end, a second end, and first and second furnace sidewalls extending from the first end to the second end;
a radiative heat exchanger section comprising an exchanger housing having an exchanger first end, an exchanger second end, and first and second exchanger sidewalls extending from the exchanger first end to the exchanger second end;
a plurality of electrically conductive reactor tubes extending through the heater housing and the exchanger housing, each of the reactor tubes defining a flowpath from an inlet end of the reactor tube to an outlet end of the reactor tube, each of the inlet ends and outlet ends having a flanged connector;
an electric circuit configured to electrically connect in series a longitudinal portion of each of the reactor tubes such that application of a voltage difference between a first end of the longitudinal portion of a first one of the reactor tubes and a second end of the longitudinal portion of a last one of the reactor tubes will cause electrical current to flow sequentially through the longitudinal portions of all of the plurality of reactor tubes, where at least a majority of the longitudinal portions are disposed in the heater housing;
where the radiative heat exchanger section is configured to, for each of the reactor tubes, reduce the temperature of the outlet end of the tube relative to the nearest first or second end of the longitudinal portion of the tube by arranging flow within adjacent reactor tubes to be in an alternating direction or by defining at least one channel between an inner wall and at least one of the first exchanger sidewall or the second exchanger sidewall, the channel being operable to absorb thermal energy from the reactor tubes through the inner wall.

2. The furnace of claim 1, where the inlet ends of the reactor tubes are disposed on the first end of the heater housing, and the outlet ends of the reactor tubes are disposed on the second end of the exchanger housing.

3. The furnace of claim 1, where the inlet ends and the outlet ends of the reactor tubes are disposed on the second end of the exchanger housing, and each reactor tube includes a U-shaped portion at the first end of the heater housing.

4. The furnace of claim 1, where the plurality of reactor tubes includes a first subset of the reactor tubes and a second subset of the reactor tubes, the first subset of the reactor tubes are disposed with their inlet ends on the first end of the heater housing and their outlet ends on the second end of the exchanger housing, and the second subset of the reactor tubes are disposed with their outlet ends on the first end of the heater housing and their inlet ends on the second end of the exchanger housing.

5. The furnace of claim 1, where the electrical circuit connects the second end of the longitudinal portion of a first one of the reactor tubes to the first end of the longitudinal portion of a second one of the reactor tubes, and connects the second end of the longitudinal portion of the second one of the reactor tubes to the first end of the longitudinal portion of a third one of the reactor tubes.

6. The furnace of claim 1, where: (a) the first exchanger sidewall defines a channel configured to receive a fluid to absorb thermal energy from the first exchanger sidewall; (b) the second exchanger sidewall defines a channel configured to receive a fluid to absorb thermal energy from the second exchanger sidewall; or (c) both (a) and (b).

7. The furnace of claim 6, where an outlet of each channel(s) is coupled in fluid communication to a steam drum.

8. The furnace of claim 1, further comprising a catalyst disposed in the reactor tubes, where the catalyst is a steam-reforming catalyst configured to cause steam and methane gas to react to form hydrogen and carbon monoxide.

9. The furnace of claim 1, where: (a) the outlet ends of the reactor tubes are coupled to an electrically conductive collection manifold, and where the reactor tubes are electrically isolated from the collection manifold; (b) the inlet ends of the reactor tubes are coupled to an electrically conductive feedstock manifold, and where the reactor tubes are electrically isolated from the feedstock manifold; or (c) both (a) and (b).

10. The furnace of claim 8, where the electrical isolation of each reactor tube is provided by one or more flanged pipe isolation joints (PIJs).

11. A method comprising:
heating a feedstock using the furnace of claim 1 by applying a voltage difference between a first end of the longitudinal portion of a first one of the reactor tubes and a last end of the longitudinal portion of a second one of the reactor tubes to cause electrical current to flow sequentially through the longitudinal portions of all of the plurality of reactor tubes;
where the voltage difference is greater than 100 volts (V); and
where the current does not flow into either of the collection manifold or the feedstock manifold.

12. The method of claim 11, where the furnace is part of a steam methane reformer system, and the feedstock comprises steam and methane.

13. The method of claim 11, where the furnace is part of a steam cracking system, and the feedstock comprises steam and at least one component selected from the list of components consisting of: naphtha, liquefied petroleum gas, and ethane.

14. The method of claim 11, wherein (a) the first exchanger sidewall defines a channel configured to receive a fluid to absorb thermal energy from the first exchanger sidewall; (b) the second exchanger sidewall defines a channel configured to receive a fluid to absorb thermal energy from the second exchanger sidewall; or (c) both (a) and (b) and where an outlet of each channel(s) is coupled in fluid communication to a steam drum.

15. The method of claim 14, further comprising:
passing H₂O through the channel(s) of the exchanger sidewall(s) to absorb thermal energy from the exchanger sidewall(s).

16. The method of claim 15, further comprising:
delivering the H₂O from the channel(s) of the exchanger sidewall(s) to the steam drum to separate liquid and steam from the H₂O.
